(19) United States
(12) Patent Application Publication
(10) Pub. No.: US 2026/0057974 A1
(43) Pub. Date: Feb. 26, 2026

(54) METHOD FOR SEARCHING FOR STRUCTURAL GENES OF GLASS

(71) Applicant: SOUTH CHINA UNIVERSITY OF TECHNOLOGY, Guangzhou (CN)

(72) Inventors: Zhongmin YANG, Guangzhou (CN); Guoquan QIAN, Guangzhou (CN)

(73) Assignee: SOUTH CHINA UNIVERSITY OF TECHNOLOGY, Guangzhou (CN)

(21) Appl. No.: 19/373,664

(22) Filed: Oct. 29, 2025

Related U.S. Application Data
(63) Continuation-in-part of application No. 17/622,867.

(30) Foreign Application Priority Data
Jun. 27, 2019 (CN) 201910566848.3

(51) Int. Cl. G16C 60/00; C03C 3/14; C03C 3/16; C03C 3/253; G16C 20/70; G16C 20/80

(52) U.S. Cl. CPC G16C 60/00; C03C 3/14; C03C 3/16; C03C 3/253; G16C 20/70; G16C 20/80

(57) ABSTRACT

The present invention relates to a method for searching for a structural gene of glass, including the following steps: determining atomic species for structure search according to the glass system; screening structure on the basis of the first principle to screen out compounds that can be formed by the interaction among each of the atoms; comparing the formation energy and the phonon spectrum of each compound to obtain stable compounds; and constructing a metastable composition diagram of a glass system according to the stable compounds, in metastable composition diagram, a micro-structural unit of a glassy compound near a target glass composition point is the structural gene of glass in the metastable glass composition diagram; designing glass properties based on the characteristics of the structural genes; and realizing the design of high-performance glasses through a hot-melt method.

### CROSS-REFERENCE TO RELATED APPLICATION

This application is a continuation-in-part application of U.S. application Ser. No. 17/622,867, filed on Dec. 27, 2021, now pending. The prior U.S. application Ser. No. 17/622,867 is a 371 of international application of PCT application serial no. PCT/CN2019/112417, filed on Oct. 22, 2019, which claims the priority benefit of China application no. 201910566848.3, entitled Method for Searching for Structural Genes of Glass and filed on 27 Jun. 2019. The entirety of each of the above mentioned patent applications is hereby incorporated by reference herein and made a part of this specification.

### TECHNICAL FIELD

The present invention relates to the research field of glass materials, and in particular to a method for searching for a structural gene of glass.

### RELATED ART

Functional glass has been widely applied in various fields of national economy, including: laser weapons, laser medicine, building components, daily life, national defense construction, biomedicines, safety protection, and the like. Therefore, the rapid research and development of functional glass are particularly significant for economic development, people's life and national security. However, for glass, such an old material which has been invented for use over thousands of years, the problem of relationship between glass composition-structure-performance has been not solved completely, which hinders the efficient and low-cost research and development of the functional glass. The research and development of glass materials are mainly performed by a trial-and-error method, which can involve long experimental periods and high costs, and thus, this approach is highly inefficient. However, with the proposal of “Materials Genome Initiative”, material research and development have been put into a brand new mode.

The United States put forward the “Materials Genome Initiative” in 2011. Afterwards, China also has carried out corresponding layout positively. The research approach of material gene engineering is a new pattern in material research and development, and is a new “thruster” for the research and development of new materials. For the material gene method, the sequential iterative method in the traditional trial-and-error method is replaced by a high-throughput concurrent iterative method, which changes from the “experiment guided by experience” mode towards the material research and development mode of “theoretical prediction in combination with experimental verification” step by step, thus achieving the objectives of “shortening R&D cycle by half, and lowering R&D cost by half”, and accelerating the “discovery-development-production-application” process of new materials. The research approach of material gene engineering has achieved great research progress in the fields, such as thermoelectric materials, lithium ion battery materials, alloy materials and aerospace materials. But due to randomness and diversity in the structure of glass materials, the application of the material gene method in glass materials is in a difficult situation. The idea of material gene is put forward by virtue of the concept of biological gene, and whether a glass material has a specific gene like a living body. Therefore, how to accurately and rapidly search for a structural gene of a glass system becomes the key technical difficulty in the field.

Based on the early-stage studies, we have found that the structure of glass is exactly similar to a glassy compound in the corresponding phase diagram thereof, and lots of performances of glass may be predicted by an adjacent glassy compound. Based on this, we put forward that a micro-structural unit of a glassy compound in the corresponding glass phase diagram is the structural gene of the glass. For a glass system with relatively complete phase diagram data, we can search for the structural gene of the glass system by referring to a large number of literature and searching phase diagram database. However, there are the following problems in the technology: (1) important data in the literature and phase diagram database often has the problems such as non-uniform experimental conditions and errors in the experimental test method, which is extremely disadvantage for the judgment and searching of a structural gene of glass; (2) the method is invalid to the glass system whose phase diagram database is relatively incomplete. Therefore, for a glass system without phase diagram data, how to accurately and rapidly search for a structural gene of a glass system becomes the key technical difficulty in the field.

### SUMMARY OF INVENTION

Based on this, it is necessary to provide a method for searching for a structural gene of glass.

The present invention provides a method for searching for a structural gene of glass, including the following steps:
determining atomic species for structure search according to components of a multicomponent glass system;
performing structural screening by a local particle swarm optimization algorithm, on the basis of the first principle of molecular dynamics, to select compounds that can be formed by the interaction between each of the atoms;
evaluating a stability of the selected compounds from previous step, namely, calculating formation energy and phonon spectrum of each compound by using a structural relaxation calculation element, and comparing the formation energy and the phonon spectrum of each compound to obtain stable compounds;
constructing a metastable composition diagram of the glass system by plotting coordinates of the stable compounds in the coordinate system according to atomic constituent ratios of the stable compound by using atoms or oxides as a coordinate vertices, and then connecting the atoms or oxides to the stable compounds following a minimum-area principle, wherein a micro-structural unit of a glassy compound near a target glass composition point is the structural gene of glass; and
calculating the properties of the multicomponent target glass through a lever-rule formula:

$${P}_{0} = {\sum }_{i = 1}^{n} {P}_{i} \times {L}_{i} ,$$

where the multicomponent glass system contains n constituents, P₀ represents property of the target glass, Pᵢ represents property of the structural gene in the target glass, and Lᵢ represents content of that structural gene in the target glass; the properties of any composition within the multicomponent glass system can be computed by virtue of the structural-gene properties, enabling glass-performance design while reducing experimental costs by 50% and shortening the development cycle by 50%; meanwhile, glass recipe can be reverse-designed through the model for a specified set of properties, weighing corresponding raw material precisely according to the glass recipe, performing glass melting, annealing and polishing, and testing property finally, thereby achieving purposeful glass design.

The present invention provides a method for searching for a structural gene of a binary glass system, including the following steps:
performing structural screening by a local particle swarm optimization algorithm, on the basis of the first principle of molecular dynamics, to select compounds that can be formed by every two or three atoms in components of a target glass;
evaluating a stability of the selected compounds from previous step, namely, calculating formation energy and phonon spectrum of each compound by using a structural relaxation calculation element, and comparing the formation energy and the phonon spectrum of the compounds respectively to obtain stable compounds;
drawing a component triangle with composition atoms of the target glass as coordinate vertices, and marking out coordinates of the stable compounds in the component triangle, followed by connecting the atoms or oxides to the stable compounds following a minimum-area principle, to obtain a metastable composition diagram of the binary glass system;
finding out component coordinates of the target glass in the metastable composition diagram of the binary glass system, wherein a micro-structural unit of a glassy compound corresponding to the two stable compounds adjacent to the component coordinates is the structural gene of the target glass; and
calculating the properties of the target glass through a binary glass lever-rule formula:

$${P}_{0} = P 1 \times L 1 + P 2 \times L 2 ,$$

where P₀ represents property of the target glass, P1 and P2 represent property of the structural gene in the target glass, and L1 and L2 represent content of that structural gene in the target glass; the properties of any composition within the binary glass system can be computed by virtue of the structural-gene properties, enabling glass-performance design while reducing experimental costs by 50% and shortening the development cycle by 50%; meanwhile, glass recipe can be reverse-designed through the model for a specified set of properties, weighing corresponding raw material precisely according to the glass recipe, performing glass melting, annealing and polishing, and testing property finally, thereby achieving purposeful glass design.

In one of the embodiment, the step of performing structural screening on the basis of the first principle of molecular dynamics is high-throughput structural screening performed by a first principle of molecular dynamics structural screening software.

In one of the embodiment, a local particle swarm optimization (PSO) algorithm is used in the high-throughput structural screening algorithm.

In one of the embodiment, the local PSO algorithm calculates 35 to 50 structures each generation, and calculates 20 to 30 generations in total.

In one of the embodiment, the high-throughput structural screening further includes structure relaxation calculation.

In one of the embodiment, the structure relaxation has a cut-off energy of 400 ev to 500 ev, and a functional is a PBE functional in Generalized Gradient Approximation.

In one of the embodiment, the method further includes a step of determining a number range of each atom according to the atomic species of the components of the target glass before the step of performing structural screening on the basis of the first principle of molecular dynamics.

In one of the embodiment, the step of respectively comparing the formation energy and the phonon spectrum of the compounds includes the followings:
constructing a bump diagram of the calculated formation energy of the compounds varying with components, and judging thermodynamically stable compounds among the compounds according to the bump diagram;
calculating a phonon spectrum of the thermodynamically stable compound, and choosing a compound free of imaginary frequency in the phonon spectrum thereof as the stable compound.

In one of the embodiment, the target glass includes one or more of laser glass, optical glass, bioglass, nuclear glass, safety glass and ware glass.

The present invention provides a method for searching for a structural gene of a ternary glass system, including the following steps:
making a combination with any two of three components of a target glass to obtain three binary composition systems, respectively performing structural screening on each of the binary composition systems according to the method for searching for a structural gene of a binary glass system to obtain corresponding stable compounds in each of the binary composition systems;
making a combination with the three components of the target glass to obtain a ternary composition system, determining a ratio between 4 atoms in the ternary composition system, and performing structural screening on the basis of the first principle to screen out compounds that can be formed by 4 atoms in the ternary composition system;
comparing the formation energy and the phonon spectrum of the compounds that can be formed by 4 atoms in the ternary composition system with the formation energy and the phonon spectrum of the stable compounds in the binary composition system, determining the stable compounds in the compounds that can be formed by 4 atoms in the ternary composition system;
drawing a component triangle with the components in the ternary composition system as vertices, and marking out all the coordinates of the stable compounds in the binary composition system and all the coordinates of the stable compounds in the ternary composition system in the component triangle, and dividing a triangular region with all the coordinates of the stable compounds as vertices and according to a principle of minimizing area to obtain a metastable composition diagram of the ternary glass system;
finding out component coordinates corresponding to the target glass in the metastable composition diagram of the ternary glass system, wherein a micro-structural unit of a glassy compound corresponding to the compound represented by three vertices of the triangular region where the component coordinates are located is a structural gene of the target glass.

In one of the embodiment, the step of comparing the formation energy and the phonon spectrum of the compounds that can be formed by 4 atoms in the ternary composition system with the formation energy and the phonon spectrum of the stable compounds in the binary composition system includes the followings:
using the stable compounds in the binary composition system as end points of components, constructing a bump diagram of the formation energy of the compounds that can be formed by 4 atoms in the ternary composition system varying with components, and judging the thermodynamically stable compounds according to the bump diagram;
calculating a phonon spectrum of the thermodynamically stable compound, and choosing a compound free of imaginary frequency in the phonon spectrum thereof as the stable compound.

In one of the embodiment, when the stable compound is not present in the compounds that can be formed by 4 atoms in the ternary composition system, all the stable compounds in the binary composition system are marked out in the component triangle only.

In one of the embodiment, when the stable compound is present in the compounds that can be formed by 4 atoms in the ternary composition system, all the stable compounds in the binary composition system and all the stable compounds in the ternary composition system are marked out in the component triangle.

Compared with the method in the prior art, the present invention has the following advantages:
(1) There are two major problems in the method for determining the gene of glass by searching for phase diagram database at present: a, important data in the literature and phase diagram database often has the problems such as non-uniform experimental conditions and errors in the experimental test method, which is extremely disadvantage for the judgment and searching of the structural gene of glass; b, the method for determining the gene of glass is invalid to the glass system whose phase diagram database is relatively incomplete. Based on the short-range order of glass, the present invention innovatively puts forward a method for searching for a structural gene of glass starting from the level of atom for the first time. The present invention first puts forward a research method for making a combination with atom-compound-glass by structural screening on the basis of the first principle of molecular dynamics; the method can effectively solve the problem of the glass system with incomplete phase diagram data and thus, is a method for rapidly and efficiently breaking a structural gene of glass. Therefore, the present invention is of great significance to the predication of composition-structure-performance of glass.
(2) In this present invention, the concept of biological gene and research approach of material gene engineering are applied in the study of functional glass to rapidly search for a structural gene of a glass system; based on the structure and properties of the structural gene of the glass system, the internal structure of glass may be realized thoroughly to break the glass structure, which is beneficial to the design of functional glass as desired and has great significance for the research and development of glass.
(3) In the metastable composition diagram of a glass system, a specific composition point of the glass system corresponds to a specific structural gene of the glass system, and the structural gene of the glass system is studied comparatively to quantitatively study the structure and performance of the target glass.
(4) The method provided by the present invention may further greatly expand the structural performance database of the compound, which is beneficial to the rapid and low-cost research and development of the functional glass.

### BRIEF DESCRIPTION OF DRAWINGS

FIG. 1 shows a flow diagram of a method for searching for a structural gene of a multicomponent glass system in one example of the present invention;

FIG. 2 shows a flow diagram of a method for searching for a structural gene of a binary glass system in one example of the present invention;

FIG. 3 shows a bump diagram of relative formation energy of a stable compound in a B₂O₃—Li₂O binary glass system in Example 1 of the present invention varying with components;

FIG. 4 shows a metastable composition diagram of the B₂O₃—Li₂O binary glass system in Example 1 of the present invention;

FIG. 5 shows a metastable composition diagram of a Li₂O—MgO—B₂O₃ ternary glass system in Example 2 of the present invention;

FIG. 6 shows a metastable composition diagram of a BaO—CaO—P₂O₅ ternary glass system in Example 3 of the present invention;

FIG. 7 shows a metastable composition diagram of the BaO—Ga₂O₃—GeO₂ ternary glass system in Example 4 of the present invention;

FIG. 8 shows a bump diagram of relative formation energy of a stable compound in a Li₂O—GeO₂ binary glass system in Example 5 of the present invention varying with components;

FIG. 9 shows a metastable composition diagram of the Li₂O—GeO₂ binary glass system in Example 5 of the present invention;

FIG. 10 shows a comparison graph of the predicted and experimental values of density and refractive index for the Li₂O—GeO₂ and Na₂O—GeO₂ binary glass systems of Example 5 of the present invention.

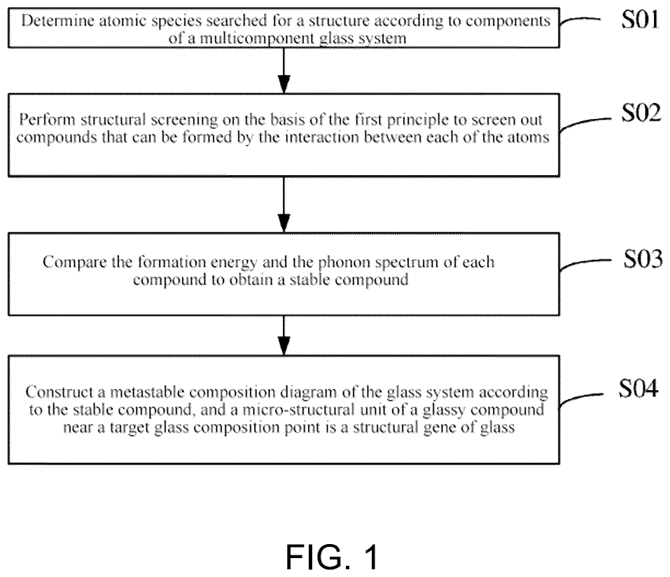

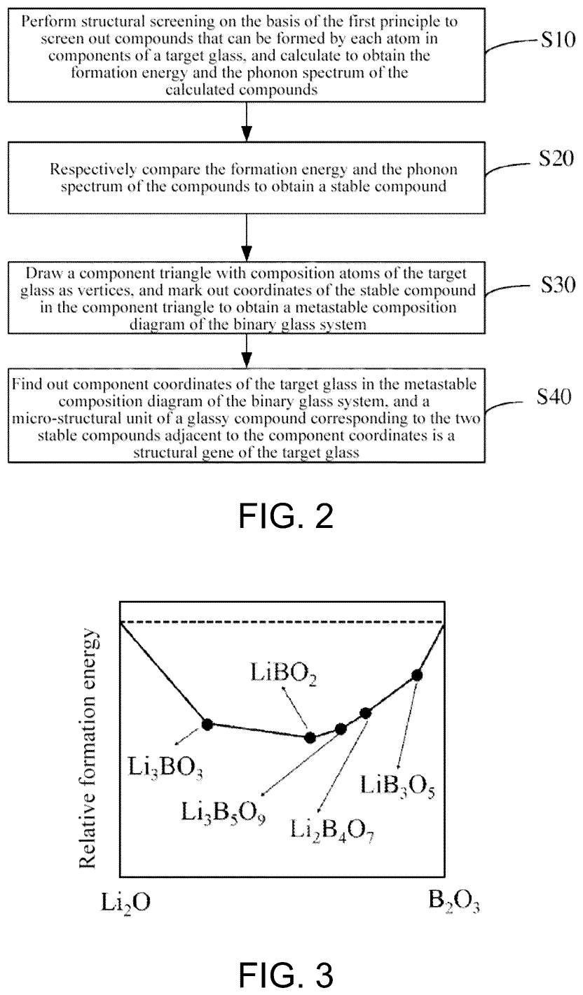

### DESCRIPTION OF EMBODIMENTS

To describe the objective, technical solution and advantages of the present invention more clearly and apparently, the present application will be further described specifically through examples and in combination with the drawings. It should be understood that detailed embodiments described herein are merely used to explain the present invention, rather than limit the present invention.

The present invention provides a method for searching for a structural gene of a multicomponent glass system, including the following steps:
S01, determining atomic species for structure search according to components of the multicomponent glass system;
S02, performing structural screening on the basis of the first principle of molecular dynamics to select compounds that can be formed by the interaction between each of the atoms;
S03, evaluating a stability of the selected compounds from previous step, namely, calculating formation energy and phonon spectrum of each compound by using a structural relaxation calculation element, and comparing the formation energy and the phonon spectrum of each compound to obtain stable compounds;
S04, taking constituent atoms of the target glass as vertices of a composition triangle, marking inside the triangle the coordinates of the stable compounds, connecting the atoms or oxides to the stable compounds following a minimum-area principle, and constructing a metastable composition diagram of the glass system, wherein a micro-structural unit of a glassy compound near a target glass composition point is a structural gene of glass.

The method for searching for the structural gene of a multicomponent glass system provided by the present invention is rapid and efficient. Based on the short-range order feature of glass, the present invention innovatively puts forward a method for searching for a structural gene by making a combination with atom-compound-glass via structural screening and on the basis of the first principle of molecular dynamics, namely, a method for searching for the structural gene of glass starting from the level of atom. Based on the structural gene, the method of the present invention may be used to realize the structure of glass more thoroughly, which lays the foundation for the in-depth prediction of glass structural performance as well as for the rapid, low-cost and efficient research and development of functional glass. Therefore, the present invention is of great significance to the predication of composition-structure-performance of glass.

In this present invention, the multicomponent glass system consists of a plurality of oxides, and the components are oxides constituting the multicomponent glass system; the binary glass system includes two components, and the ternary glass system includes three components, for example, Li₂O—B₂O₃ binary glass system consists of Li₂O, B₂O₃, and BaO—Ga₂O₃—GeO₂ ternary glass system consists of BaO, Ga₂O₃, and GeO₂.

In this present invention, the compound includes a plurality of different compounds, and the different compounds include compounds with different atomic compositions, and further include compounds with the same atomic composition and different structures.

The present invention provides a method for searching for a structural gene of a binary glass system, including the following steps:
S10, performing structural screening on the basis of the first principle of molecular dynamics to select compounds that can be formed by every two or three atoms in components of a target glass;
S20, evaluating a stability of the selected compounds from previous step, namely, calculating formation energy and phonon spectrum of each compound by using a structural relaxation calculation element, and comparing the formation energy and the phonon spectrum of the compounds to obtain stable compounds respectively;
S30, drawing a component triangle with composition atoms of the target glass as coordinate vertices, and marking out coordinates of the stable compounds in the component triangle, followed by connecting the atoms or oxides to the stable compounds following a minimum-area principle, to obtain a metastable composition diagram of the binary glass system;
S40, finding out component coordinates of the target glass in the metastable composition diagram of the binary glass system, wherein a micro-structural unit of a glassy compound corresponding to the two stable compounds adjacent to the component coordinates is a structural gene of the target glass.

In one embodiment, the step of performing structural screening on the basis of the first principle of molecular dynamics is high-throughput structural screening performed by a first principle of molecular dynamics structural screening software, for example, CALYPSO and VASP.

In one embodiment, a local particle swarm optimization (PSO) algorithm is used in the high-throughput structural screening algorithm, preferably, the local PSO algorithm calculates 35 to 50 structures each generation, and calculates 20 to 30 generations in total.

In one embodiment, the high-throughput structural screening further includes structure relaxation calculation, preferably, the structure relaxation calculation has a cut-off energy of 400 ev to 500 ev, and a functional is a PBE functional in Generalized Gradient Approximation.

In one embodiment, the method further includes a step S00, determining a number range of each atom according to the atomic species of the components of the target glass before the step of performing structural screening on the basis of the first principle.

In one embodiment, in the step S20, the step of comparing the formation energy and the phonon spectrum of each compound includes the followings:
S22, constructing a bump diagram of the calculated formation energy of the compounds varying with components, and judging thermodynamically stable compounds among the compounds according to the bump diagram;
S24, calculating a phonon spectrum of the thermodynamically stable compound, and choosing a compound free of imaginary frequency in the phonon spectrum thereof (namely, a dynamically stable compound), as the stable compound.

The step of comparing the formation energy and phonon spectrum of each compound includes a comparison of the formation energy and the phonon spectrum between different compounds, and further includes a comparison of the formation energy and the phonon spectrum between different structures of a same compound.

In the step S30, the component triangle is a triangle drawn according to a component representation method of a multicomponent phase diagram, and also may be called a concentration triangle. A parallel line for each side of the component triangle is respectively drawn through any point in the component triangle, and a line segment of the parallel line cut in each side of the component triangle respectively represents a concentration or a ratio of each component of the point. The coordinate is the corresponding point of a compound with specific composition in the component triangle. In the step S40, the component coordinates are corresponding points of components of the target glass in the component triangle.

In one embodiment, the target glass includes one or more of laser glass, optical glass, bio-glass, nuclear glass, safety glass and ware glass.

The present invention further provides a method for searching for a structural gene of a ternary glass system in examples, including the following steps:
S100, making a combination with any two of three components of a target glass to obtain three binary composition systems, respectively performing structural screening on each of the binary composition systems according to the method for searching for a structural gene of a binary glass system to obtain the corresponding stable compound in each of the binary composition systems;
S200, making a combination with the three components of the target glass to obtain a ternary composition system, determining a ratio between 4 atoms in the ternary composition system, and performing structural screening on the basis of the first principle to screen out compounds that can be formed by 4 atoms in the ternary composition system;
S300, comparing the formation energy and the phonon spectrum of the compounds that can be formed by 4 atoms in the ternary composition system with the formation energy and the phonon spectrum of the stable compounds in the binary composition system, determining a stable compound in the compounds that can be formed by 4 atoms in the ternary composition system;
S400, drawing a component triangle with the components in the ternary composition system as vertices, and marking out all the coordinates of the stable compounds in the binary composition system and all the coordinates of the stable compounds in the ternary composition system in the component triangle, and dividing a triangular region with all the coordinates of the stable compounds as vertices and according to a principle of minimizing area to obtain a metastable composition diagram of the ternary glass system;
S500, finding out component coordinates corresponding to the target glass in the metastable composition diagram of the ternary glass system, where a micro-structural unit of a glassy compound corresponding to the compound represented by three vertices of the triangular region where the component coordinates are located is a structural gene of the target glass.

In one example, the step S300 of comparing the formation energy and the phonon spectrum of the compounds that can be formed by 4 atoms in the ternary composition system with the formation energy and the phonon spectrum of the stable compounds in the binary composition system includes the followings:
S320, using the stable compounds in the binary composition system as end points of components, constructing a bump diagram of the formation energy of the compounds that can be formed by 4 atoms in the ternary composition system varying with components, and judging the thermodynamically stable compounds according to the bump diagram;
S340, calculating a phonon spectrum of the thermodynamically stable compound, and choosing a compound free of imaginary frequency in the phonon spectrum thereof as the stable compound.

In one example, when the stable compound is not present in the compounds that can be formed by 4 atoms in the ternary composition system, step S400, all the stable compounds in the binary composition system are marked out in the component triangle only.

In one example, when the stable compound is present in the compounds that can be formed by 4 atoms in the ternary composition system, step S400, all the stable compounds in the binary composition system and all the stable compounds in the ternary composition system are marked out in the component triangle.

The method provided by the examples of the present invention is used to search for a structural gene of a glass system by virtue of the concept of biological gene and research approach of material gene engineering. The sequential iterative method in the traditional trial-and-error method is replaced by a high-throughput concurrent iterative method, which changes from the “experiment guided by experience” mode towards the material research and development mode of “theoretical prediction in combination with experimental verification” step by step, thus achieving the objectives of “shortening R&D cycle by half, and lowering R&D cost by half”, and accelerating the “discovery-development-production-application” process of new materials.

The metastable composition diagram of the binary glass system and the metastable composition diagram of the ternary glass system reflect the real composition and structure of glass; the glass composition points may achieve one-to-one correspondence in the diagram. In the metastable composition diagram of the binary glass system and the metastable composition diagram of the ternary glass system, a micro-structural unit of a glassy compound corresponding to the two stable compounds adjacent to the component coordinate of the target glass or the compound represented by three vertices of the triangular region where the component coordinates are located is the structural gene of the target glass.

The structural gene of the glass system contains the polyhedron coordination situation with the same target glass, reflects the short-range structure of glass and determines the property of glass. The composition points of the glass system may achieve one-to-one correspondence in the metastable composition diagram of the glass system.

### Example 1 Binary System

Target Glass: 41 Mol % Li₂O—59 Mol % B₂O₃

A searching number range of each atom in B, Li and O was set, and the number of B atoms was 0-8, the number of Li atoms was 0-3, and the number of O atoms was 1-13;
according to a ratio of atoms, structural screening was performed by a first principle structural screening software CALYPSO; structure evolution was performed by a local PSO algorithm, and 35 structures were produced in each generation; the screened structures were subjected to structure relaxation by a first principle calculation software VASP, and the cut-off energy was 600 ev, and a functional was a PBE functional in Generalized Gradient Approximation, so as to obtain compounds that could be formed: B₂O₃, BO₆, B₆O, LiB₃O₅, Li₃BsO₉, Li₂B₄O₇, LiBO₂, Li₃BO₃, Li₂BsO₁₃, LiO, Li₂O, Li₃O, and the formation energy of these compounds.

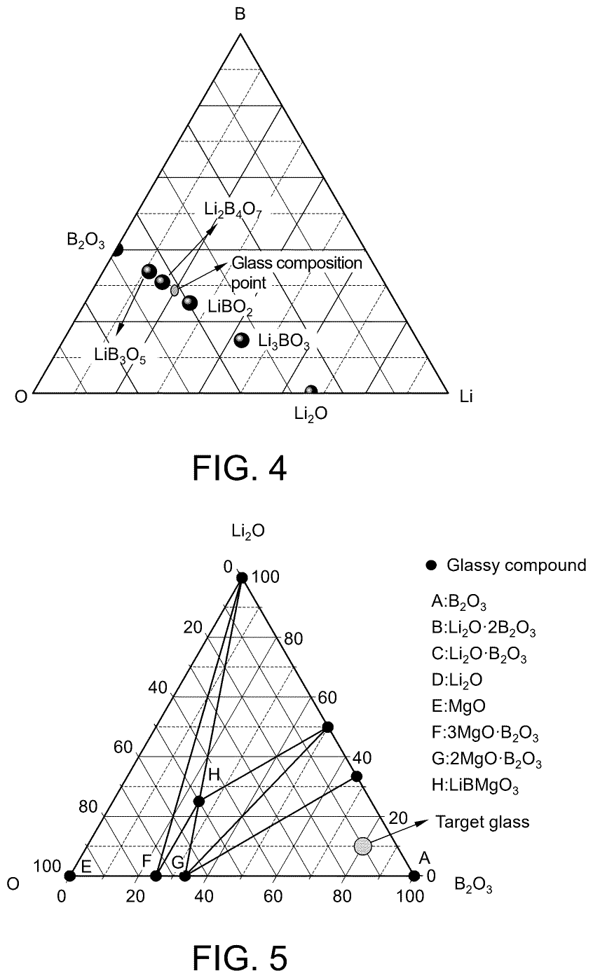

A bump diagram of formation energy varying with components was constructed on the basis of the formation energy of the compounds, as shown in FIG. 3; thermodynamically stable compounds among the compounds were judged Li₃BsO₉, B₂O₃, LiB₃O₅, Li₂B₄O₇, LiBO₂, Li₃BO₃, Li₂O according to the bump diagram;
a phonon spectrum of the thermodynamically stable compound was calculated, and a compound free of imaginary frequency in the phonon spectrum thereof was chosen to be the stable compound, including B₂O₃, LiB₃O₅, Li₂B₄O₇, LiBO₂, Li₃BO₃, Li₂O;
a component triangle was drawn with three atoms B, Li and O as vertices, and coordinates of B₂O₃, LiB₃O₅, Li₂B₄O₇, LiBO₂, Li₃BO₃, Li₂O were marked out in the component triangle to obtain a metastable composition diagram of the B₂O₃—Li₂O binary glass system, as shown in FIG. 4;
the component coordinates of the target glass were found out in FIG. 4, and the coordinates of the target glass were located between LiBO₂ and Li₂B₄O₇, the structural gene of the glass composed of 41 mol % Li₂O—59 mol % B₂O₃ was glassy LiBO₂ and Li₂B₄O₇.

### Example 2 Ternary System

Target Glass: 10 Mol % Li₂O—10 Mol % MgO—80 Mol % B₂O₃

Any two of components Li₂O, MgO and B₂O₃ were combined to obtain a MgO—B₂O₃ binary composition system, a Li₂O—B₂O₃ binary composition system and a Li₂O—MgO binary composition system; steps S00-S60 were performed to respectively obtain the stable compounds in the MgO—B₂O₃ binary composition system, including MgO, 2MgO·B₂O₃, 3MgO·B₂O₃, and B₂O₃; the stable compounds in Li₂O—B₂O₃ binary composition system, including B₂O₃, Li₂O·2B₂O₃, Li₂O B₂O₃, and Li₂O; and the stable compounds in Li₂O—MgO binary composition system, including Li₂O and MgO;
components Li₂O, MgO and B₂O₃ were combined to obtain a Li₂O—MgO—B₂O₃ ternary composition system; the searching number range of the 4 atoms in the ternary composition system was as follows: L1: 1-5, Mg: 1-5, B: 1-5, and O: 1-10; a first principle structural screening software and calculation software were utilized for high-throughput structural screening to screen out the compounds that could be formed by the 4 atoms of L1, Mg, B and O, where the existing compounds included Li₂BMgO₃ and LiBMgO₃, and the formation energy and phonon spectrum were calculated.

The formation energy and the phonon spectrum of the compounds that could be formed by the 4 atoms of L1, Mg, B and O were compared with the formation energy and the phonon spectrum of the stable compounds in the metastable composition diagram of the MgO—B₂O₃ binary composition system and the stable compounds in the metastable composition diagram of the Li₂O—B₂O₃ binary composition system. Based on the comparison result, LiBMgO₃ could be stable in the compounds that could be formed by the 4 atoms of L1, Mg, B and O;
a component triangle was drawn with Li₂O, MgO and B₂O₃ as vertices to mark out coordinates (A: B₂O₃, B: Li₂O·2B₂O₃, C: Li₂O·B₂O₃, D: Li₂O, E: MgO, F: 3MgO·B₂O₃, G; 2MgO·B₂O₃, H: LiBMgO₃) of all the stable compounds in the component triangle; a triangular region was drawn with A, B, C, D, E, F, G and H as vertices and according to a principle of minimizing area to obtain a metastable composition diagram of the ternary glass system, as shown in FIG. 5.

The component coordinate of the target glass were found out in FIG. 5, and the coordinate was located within the ΔABG, and the structural gene of the target glass was glassy B₂O₃, Li₂O·2B₂O₃ and 2MgO·B₂O₃.

### Example 3 Ternary System

Target Glass: 10 Mol % BaO—10 Mol % CaO—80 Mol % P₂O₅

Any two of components BaO, CaO and P₂O₅ were combined to obtain a BaO—CaO binary composition system, a BaO—P₂O₅ binary composition system and a CaO—P₂O₅ binary composition system; steps S00-S60 were performed to respectively obtain the stable compounds in the BaO—CaO binary composition system, including BaO, and CaO; the stable compounds in BaO—P₂O₅ binary composition system, including BaO, BaO·P₂O₅, 2BaO·P₂O₅, P₂O₅; and the stable compounds in CaO—P₂O₅ binary composition system, including CaO, CaO·P₂O₅, CaO·2P₂O₅, and P₂O₅;
components BaO, CaO and P₂O₅ were combined to obtain a BaO—CaO—P₂O₅ ternary composition system; the searching number range of the 4 atoms in the ternary composition system was as follows: Ba: 1-5, Ca: 1-5, P: 1-5, and O: 1-10; a first principle structural screening software and calculation software were utilized for high-throughput structural screening to screen out the compounds that could be formed by the 4 atoms of Ba, Ca, P, and O, and the formation energy and phonon spectrum thereof were calculated.

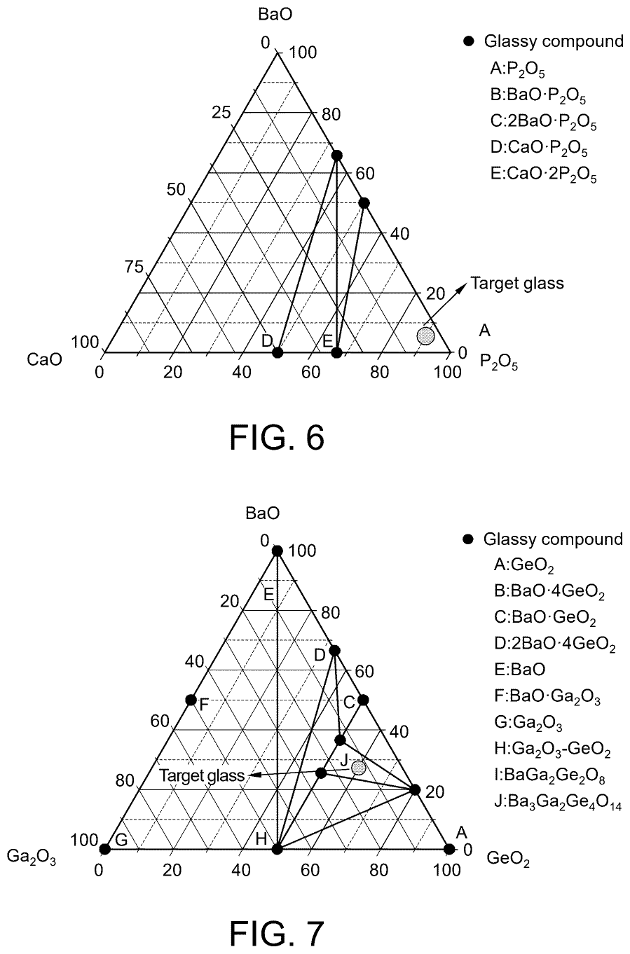

The formation energy and the phonon spectrum of the compounds that could be formed by the 4 atoms of Ba, Ca, P, and O were compared with the formation energy and the phonon spectrum of the stable compounds in the metastable composition diagram of the BaO—CaO binary composition system and the stable compounds in the metastable composition diagram of the BaO—P₂O₅ binary composition system, and the stable compounds in the metastable composition diagram of the CaO—P₂O₅ binary composition system. Based on the comparison result, there was no stable compound in the compounds that could be formed by the 4 atoms of Ba, Ca, P, and O;
a component triangle was drawn with BaO, CaO and P₂O₅ as vertices to mark out coordinates (A: P₂O₅, B: BaO·P₂O₅, C: 2BaO·P₂O₅, D: CaO·P₂O₅, E: CaO·2P₂O₅) of all the stable compounds in the component triangle; a triangular region was drawn with A, B, C, D, and E as vertices and according to a principle of minimizing area to obtain a metastable composition diagram of the ternary glass system, as shown in FIG. 6.

The component coordinate of the target glass was found out in FIG. 6, and the coordinate was located within the ΔABE, and the structural gene of the target glass was glassy P₂O₅, BaO·P₂O₅ and CaO·2P₂O₅.

### Example 4 Ternary System

Target Glass: 27 Mol % BaO—13 Mol % Ga₂O₃—60 Mol % GeO₂

Any two of components BaO, Ga₂O₃ and GeO₂ were combined to obtain a BaO—Ga₂O₃ binary composition system, a BaO—GeO₂ binary composition system and a Ga₂O₃—GeO₂ binary composition system; steps S00-S60 were performed to respectively obtain the stable compounds in the BaO—Ga₂O₃ binary composition system, including BaO, BaO·Ga₂O₃ and Ga₂O₃; the stable compounds in BaO—GeO₂ binary composition system, including BaO, BaO·4GeO₂, BaO·GeO₂, 2BaO·GeO₂ and GeO₂; and the stable compounds in Ga₂O₃—GeO₂ binary composition system, including Ga₂O₃, Ga₂O₃·GeO₂, and GeO₂;
components BaO, Ga₂O₃ and GeO₂ were combined to obtain a BaO—Ga₂O₃—GeO₂ ternary composition system; the searching number range of the 4 atoms in the ternary composition system were as follows: Ba: 1-5, Ga: 1-5, Ge: 1-6, and O: 1-15; a first principle structural screening software and calculation software were utilized for high-throughput structural screening to screen out the compounds that could be formed by the 4 atoms of Ba, Ga, Ge and O, and the formation energy and phonon spectrum thereof were calculated.

The formation energy and the phonon spectrum of the compounds that could be formed by the 4 atoms of Ba, Ga, Ge and O were compared with the formation energy and the phonon spectrum of the stable compounds in the metastable composition diagram of the BaO—Ga₂O₃ binary composition system and the stable compounds in the metastable composition diagram of the BaO—GeO₂ binary composition system, and the stable compounds in the metastable composition diagram of the Ga₂O₃—GeO₂ binary composition system. Based on the comparison result, the stable compounds in the compounds that could be formed by the 4 atoms of Ba, Ga, Ge and O included BaGa₂Ge₂O₈ and Ba₃Ga₂Ge₄O₁₄;
a component triangle was drawn with BaO, Ga₂O₃ and GeO₂ as vertices to mark out coordinates (A: GeO₂, B: BaO·4GeO₂, C: BaO·GeO₂, D: 2BaO·GeO₂, E: BaO, F: BaO·Ga₂O₃, G: Ga₂O₃, H: Ga₂O₃·GeO₂, I: BaGa₂Ge₂O₈, J: Ba₃Ga₂Ge₄O₁₄) of all the stable compounds in the component triangle; a triangular region was drawn with A, B, C, D, E, F, G, H, I, and J as vertices and according to a principle of minimizing area to obtain a metastable composition diagram of the ternary glass system, as shown in FIG. 7.

The component coordinate of the target glass was found out in FIG. 7, and the coordinate was located within the ΔBIJ, and the structural gene of the target glass was glassy BaO·4GeO₂, BaGa₂Ge₂O₈ and Ba₃Ga₂Ge₄O₁₄.

### Example 5 Li₂O-GeO₂Binary System

Target Glass: x Mol % Li₂O-y Mol % GeO₂

A searching number range of each atom in Ge, Li and O was set, and the number of Ge atoms was 0-8, the number of Li atoms was 0-8, and the number of O atoms was 1-10;
according to a ratio of each two or three atoms, structural screening was performed by a first principle structural screening software CALYPSO; structure evolution was performed by a local PSO algorithm, and 35 structures were produced in each generation; the screened structures were subjected to structure relaxation by a first principle calculation software VASP, and the cut-off energy was 500 ev, and a functional was a PBE functional in Generalized Gradient Approximation (GGA), so as to obtain compounds that could be formed: GeO₂, Li₂O·7GeO₂, Li₂O·4GeO₂, 3Li₂O·8GeO₂, Li₂O·2GeO₂, and Li₂O; and
the formation energy of these compounds.

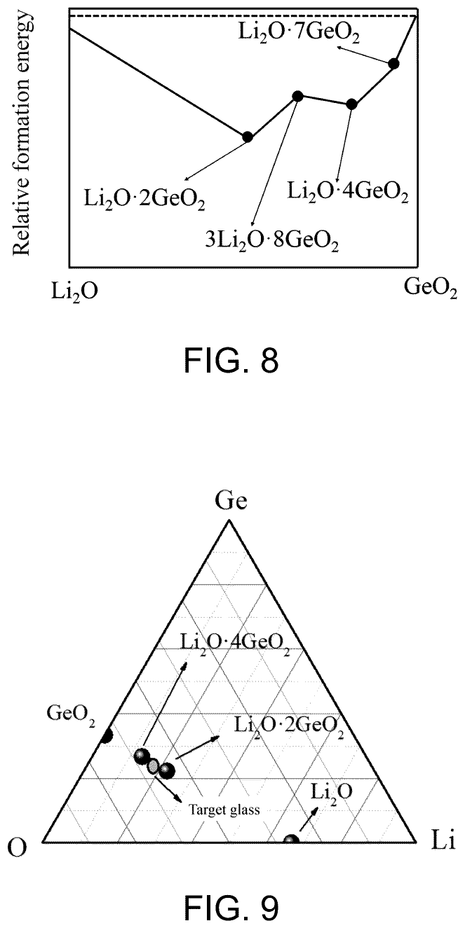

A bump diagram of formation energy varying with components was constructed on the basis of the formation energy of the compounds, as shown in FIG. 8; thermodynamically stable compounds among the compounds were judged GeO₂, Li₂O·7GeO₂, Li₂O·4GeO₂, Li₂O·2GeO₂, and Li₂O according to the bump diagram;
a phonon spectrum of the thermodynamically stable compound was calculated, and a compound free of imaginary frequency in the phonon spectrum thereof was chosen to be the stable compound, including GeO₂, Li₂O·4GeO₂, Li₂O·2GeO₂, and Li₂O;
a component triangle was drawn with three atoms Ge, Li and O as vertices, and coordinates of GeO₂, Li₂O·4GeO₂, Li₂O·2GeO₂, and Li₂O were marked out in the component triangle to obtain a metastable composition diagram of the Li₂O—GeO₂ binary glass system, as shown in FIG. 9;
when x=25.7 and y=74.3, the target glass is 25.7 mol % Li₂O—74.3 mol % GeO₂, locating a composition coordinate of the target glass in FIG. 9 where the composition coordinate falls between Li₂O·2GeO₂ and Li₂O·4GeO₂ in the FIG. 9, therefore a glassy form of Li₂O·2GeO₂ and Li₂O·4GeO₂ are the structural genes of the glass whose composition is 25.7 mol % Li₂O—74.3 mol % GeO₂;
calculating the density and refractive index of the target glass through a binary glass lever-rule formula:

$${P}_{0} = P 1 \times L 1 + P 2 \times L 2 ,$$

where P1 is a density or refractive index of Li₂O·2GeO₂ and P2 is a density or refractive index of Li₂O·4GeO₂, and experimental values for the densities or refractive indices of all glassy compounds in the Li₂O—GeO₂ and Na₂O—GeO₂ binary systems are listed in Table 1.

L1 is a content of Li₂O·2GeO₂ in the target glass and L2 is a content of Li₂O·4GeO₂ in the target glass; calculation gives L1=42.75 and L2=57.25. Inserting the densities of Li₂O·2GeO₂ and Li₂O·4GeO₂ from the Table 1 as P1 and P2 into the formula yields a predicted density P₀=3.8523, while an experimental density of the target glass 25.7 mol % Li₂O—74.3 mol % GeO₂ is 3.8612, and the relative error of the predicted density and the experimental density is within 5%. Likewise, inserting the refractive indices of Li₂O·2GeO₂ and Li₂O·4GeO₂ from the Table 1 as P1 and P2 into the formula yields a predicted refractive index of the target glass 25.7 mol % Li₂O—74.3 mol % GeO₂ is 1.694.

TABLE 1

| Glassy Compounds | Density (g/cm³) | Refractive Index |
|---|---|---|
| GeO₂ | 3.667 | 1.608 |
| Li₂O•2GeO₂ | 3.51 | 1.657 |
| Li₂O•4GeO₂ | 4.108 | 1.721 |
| 2Na₂O•9GeO₂ | 4.10 | 1.683 |
| Na₂O•2GeO₂ | 3.58 | 1.630 |
| Na₂O•GeO₂ | 3.31 | — |

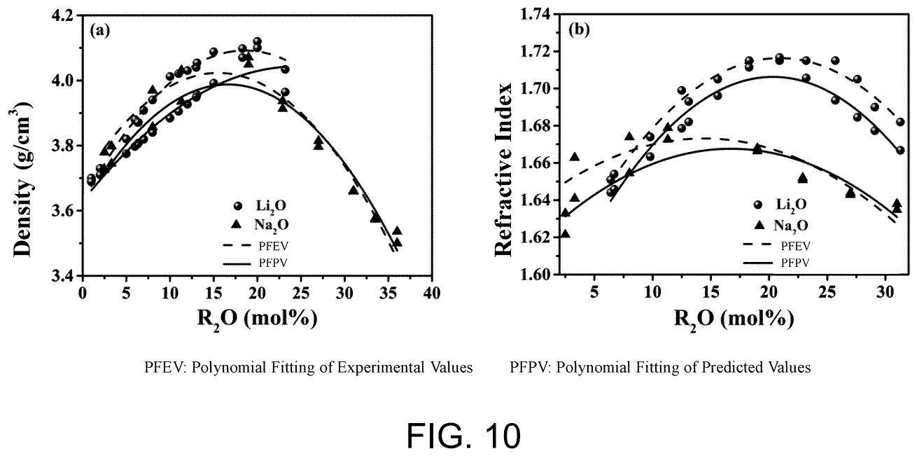

For a series of different x and y values—i.e., for Li₂O—GeO₂ binary glasses of various compositions—the predicted densities and refractive indices were calculated and compared with the experimentally measured densities and refractive indices of the corresponding Li₂O—GeO₂ binary glasses. The results are shown in FIG. 10. From FIG. 10 it can be seen that, relative to the experimental values, the relative error of the predicted densities and refractive indices obtained by the above method is within 5%, demonstrating that the proposed method for predicting the density and refractive index of binary glass systems is effective. Glass densities were measured by a water-immersion method, and refractive indices were measured with a Metricon 2010 prism-coupling instrument. For any desired set of properties, glass recipe can be reverse-designed through the model, weighing corresponding raw material precisely according to the glass recipe, performing glass melting, annealing and polishing, and testing property finally, thereby achieving purposeful glass design.

As shown in Examples 1 and 2, the method is generalized to the ternary from the binary; based on the similar step, the method provided by the present invention may be further generalized to the quaternary, quinary and even more-component glass systems.

Each technical feature of the above examples may be in any combination; to achieve brief description, all the possible combinations of each technical feature of the above examples are not described one by one. But as long as the combinations of these technical features are not contradictory, the combinations should be regarded to fall within the scope of the description.

The above examples merely express several embodiments of the present invention, and are described more specifically and particularly, but are thus not construed as limiting the scope of the invention. It should be indicated that a person skilled in the art may further make several transformations and improvements within the concept of the present invention, and these fall within the protection scope of the present invention. Therefore, the protection scope of the present invention shall be subjected to the claims attached herein.

What is claimed is:

1. A method for searching for a structural gene of a multicomponent glass system in a glass system, wherein the method comprises structure-search software design, algorithm selection, computational modules for comparison and composition-map construction, and compound-stability evaluation, comprising the following steps:
determining atomic species for structure search according to components of the multicomponent glass system;
performing structural screening by a local particle swarm optimization algorithm, on the basis of a first principle of molecular dynamics, to select compounds that can be formed by the interaction between each of the atoms;
evaluating a stability of the selected compounds from previous step, namely, calculating formation energy and phonon spectrum of each compound by using a structural relaxation calculation element, and comparing the formation energy and the phonon spectrum of each compound to obtain stable compounds; and
constructing a metastable composition diagram of the glass system by plotting coordinates of the stable compounds in the coordinate system according to atomic constituent ratios of the stable compounds by using atoms or oxides as coordinate vertices, and then connecting the atoms or oxides to the stable compounds following a minimum-area principle, wherein a micro-structural unit of a glassy compound near a target glass composition point is the structural gene of glass; and
calculating the properties of the multicomponent target glass through a lever-rule formula:
$${P}_{0} = {\sum }_{i = 1}^{\overline{n}} {P}_{i} \times {L}_{i} ,$$
where the multicomponent glass system contains n constituents, P₀ represents property of the target glass, Pᵢ represents property of the structural gene in the target glass, and Lᵢ represents content of that structural gene in the target glass; the properties of any composition within the multicomponent glass system can be computed by virtue of the structural-gene properties, enabling glass-performance design while reducing experimental costs by 50% and shortening the development cycle by 50%; meanwhile, glass recipe can be reverse-designed through the model for a specified set of properties, weighing corresponding raw material precisely according to the glass recipe, performing glass melting, annealing and polishing, and testing property finally, thereby achieving purposeful glass design.

2. A method for searching for a structural gene of a binary glass system, comprising the following steps:
performing structural screening by a local particle swarm optimization algorithm, on the basis of a first principle of molecular dynamics, to select compounds that can be formed by each atom in components of a target glass;
evaluating a stability of the selected compounds from previous step, namely, calculating formation energy and phonon spectrum of each compound by using a structural relaxation calculation element, and comparing the formation energy and the phonon spectrum of the compounds respectively to obtain stable compounds;
drawing a component triangle with composition atoms of the target glass as coordinate vertices, and marking out coordinates of the stable compounds in the component triangle, followed by connecting the atoms or oxides to the stable compounds following a minimum-area principle, to obtain a metastable composition diagram of the binary glass system; and
finding out component coordinates of the target glass in the metastable composition diagram of the binary glass system, wherein a micro-structural unit of a glassy compound corresponding to the two stable compounds adjacent to the component coordinates is the structural gene of the target glass; and
calculating the properties of the target glass through a binary glass lever-rule formula:
$${P}_{0} = P 1 \times L 1 + P 2 \times L 2 ,$$
where P₀ represents property of the target glass, P1 and P2 represent property of the structural gene in the target glass, and L1 and L2 represent content of that structural gene in the target glass; the properties of any composition within the binary glass system can be computed by virtue of the structural-gene properties, enabling glass-performance design while reducing experimental costs by 50% and shortening the development cycle by 50%; meanwhile, glass recipe can be reverse-designed through the model for a specified set of properties, weighing corresponding raw material precisely according to the glass recipe, performing glass melting, annealing and polishing, and testing property finally, thereby achieving purposeful glass design.

3. The method for searching for a structural gene of a binary glass system according to claim 2, wherein the step of performing structural screening on the basis of the first principle of molecular dynamics is a high-throughput structural screening performed by a first principle of molecular dynamics structural screening software component.

4. The method for searching for a structural gene of a binary glass system according to claim 3, wherein the local particle swarm optimization (PSO) algorithm is used in the high-throughput structural screening algorithm.

5. The method for searching for a structural gene of a binary glass system according to claim 4, wherein the local PSO algorithm calculates 35 to 50 structures each generation, and calculates 20 to 30 generations in total.

6. The method for searching for a structural gene of a binary glass system according to claim 3, wherein the high-throughput structural screening further comprises structure relaxation calculation.

7. The method for searching for a structural gene of a binary glass system according to claim 6, wherein the structure relaxation has a cut-off energy of 400 ev to 500 ev, and a functional is a PBE functional in Generalized Gradient Functional.

8. The method for searching for a structural gene of a binary glass system according to claim 2, wherein the method further comprises a step of determining a number range of each atomic structure screening according to the atomic species of the components of the target glass before the step of performing structural screening on the basis of the first principle of molecular dynamics.

9. The method for searching for a structural gene of a binary glass system according to claim 2, wherein the step of respectively comparing the formation energy and the phonon spectrum of the compounds comprises the followings:
constructing a bump diagram of the calculated formation energy of the compounds varying with components, and judging thermodynamically stable compounds among the compounds according to the bump diagram; and
calculating a phonon spectrum of the thermodynamically stable compound, and choosing a compound free of imaginary frequency in the phonon spectrum thereof as the stable compound.

10. The method for searching for a structural gene of a binary glass system according to claim 2, wherein the target glass comprises one or more of laser glass, optical glass, bio-glass, nuclear glass, safety glass, and ware glass.

11. A method for searching for a structural gene of a ternary glass system, comprising the following steps:
making a combination with any two of three components of a target glass to obtain three binary composition systems, respectively performing structural screening on each of the binary composition systems according to the method for searching for a structural gene of a binary glass system according to claim 2 to obtain the corresponding stable compound in each of the binary composition systems;
making a combination with the three components of the target glass to obtain a ternary composition system, determining a ratio between each atom in the ternary composition system, and performing structural screening on the basis of the first principle to screen out compounds that can be formed by each atom in the ternary composition system;
comparing the formation energy and the phonon spectrum of the compounds that can be formed by each atom in the ternary composition system with the formation energy and the phonon spectrum of the stable compounds in the binary composition system, determining a stable compound in the compounds that can be formed by each atom in the ternary composition system;
drawing a component triangle with the components in the ternary composition system as vertices, and marking out all the coordinates of the stable compounds in the binary composition system and all the coordinates of the stable compounds in the ternary composition system in the component triangle, and dividing a triangular region with all the coordinates of the stable compounds as vertices and according to a principle of minimizing area to obtain a metastable composition diagram of the ternary glass system; and
finding out component coordinates corresponding to the target glass in the metastable composition diagram of the ternary glass system, wherein a micro-structural unit of a glassy compound corresponding to the compound represented by three vertices of the triangular region where the component coordinates are located is a structural gene of the target glass.

12. The method for searching for a structural gene of a ternary glass system according to claim 11, wherein the step of comparing the formation energy and the phonon spectrum of the compounds that can be formed by each atom in the ternary composition system with the formation energy and the phonon spectrum of the stable compounds in the binary composition system comprises the followings:
using the stable compounds in the binary composition system as end points of components, constructing a bump diagram of the formation energy of the compounds that can be formed by each atom in the ternary composition system varying with components, and judging the thermodynamically stable compounds according to the bump diagram; and
calculating a phonon spectrum of the thermodynamically stable compound, and choosing a compound free of imaginary frequency in the phonon spectrum thereof as the stable compound.

13. The method for searching for a structural gene of a ternary glass system according to claim 11, wherein when the stable compound is not present in the compounds that can be formed by each atom in the ternary composition system, all the stable compounds in the binary composition system are marked out in the component triangle only.

14. The method for searching for a structural gene of a ternary glass system according to claim 11, wherein when the stable compound is present in the compounds that can be formed by each atom in the ternary composition system, all the stable compounds in the binary composition system and all the stable compounds in the ternary composition system are marked out in the component triangle.
